(19) United States
(12) Patent Application Publication
(10) Pub. No.: US 2026/0192052 A1
(43) Pub. Date: Jul. 9, 2026

(54) SYSTEMS, DEVICES, AND METHODS FOR DELIVERY OF THERAPIES INCLUDING STABILIZING AGENTS

(71) Applicant: RX BANDZ, INC., Locust Valley, NY (US)

(72) Inventor: Stephen Harhen, Rumson, NJ (US)

(21) Appl. No.: 19/131,030

(22) PCT Filed: Nov. 20, 2023
(86) PCT No.: PCT/US2023/080526

§ 371 Date: May 19, 2025

Related U.S. Application Data
(60) Provisional application No. 63/426,633, filed on Nov. 18, 2022.

(51) Int. Cl. A61M 5/315; A61M 5/20

(52) U.S. Cl. CPC A61M 5/31511; A61M 5/2033; A61M 2005/2073

(57) ABSTRACT

Systems, devices, and methods described herein relate to delivery of therapeutic agents. Such systems and devices can include needle-based delivery systems, in which a stabilizing agent is incorporated into the systems for controlling gas transmission into and out of the primary drug container of the therapeutic agent.

### CROSS-REFERENCE TO RELATED APPLICATIONS

This application claims priority to U.S. Provisional Patent Application No. 63/426,633, filed Nov. 18, 2022, and titled “SYSTEMS, DEVICES, AND METHODS FOR DELIVERY OF THERAPIES INCLUDING ANTIOXIDANTS,” the disclosure of which is incorporated by reference in its entirety.

### TECHNICAL FIELD

Systems, devices, and methods described herein generally relate to the delivery of therapeutic substances, and in particular, to the delivery of therapeutic substances using needle-based delivery systems with stabilizing agents into the hydraulic intermediary fluid of the autoinjector for the purpose of prolonging the shelf-life of the therapeutic substances in the primary drug container.

### BACKGROUND

Devices have been used to administer therapies under emergency conditions, such as, for example, administering epinephrine to counteract the effects of a severe allergic reaction (e.g., anaphylaxis) or naloxone for opioid overdose. Devices have also been described for use in administering therapies to treat disease, such as, for example, anti-arrhythmic medications and selective thrombolytic agents during a heart attack. One common type of delivery device is an auto-injector. Auto-injectors offer an alternative to manually operated syringes for administering therapeutic agents into subjects in need thereof, or allowing subjects in need thereof to self-administer medications.

Auto-injectors can be the preferred method of delivery for many therapeutics due to their ease of use by both trained and untrained professionals. Additionally, the speed of delivery and dose accuracy can be desired for quick administration. Auto-injectors, however, may sometimes be carried by individuals for long periods of time. For example, individuals with known allergies may carry an epinephrine auto-injector on their person in anticipation of its use in the event of an allergic reaction. Auto-injectors can store therapeutic substances such as drugs for extended periods of time, but the shelf-life of most drugs are usually about 12 months to about 3 years. For example, epinephrine auto-injectors on the market today usually have an expiration date of about 18 months from manufacture. Such limited shelf-life requires users to constantly purchase new auto-injectors, as old ones expire and become less effective. Accordingly, it can be desirable to have improved solutions for prolonging the shelf-life and effectiveness of therapeutic substances, e.g., auto-injectors and other drug delivery systems designed to enhance drug shelf-life.

### BRIEF DESCRIPTION

Systems, devices, and methods described herein relate to drug or therapeutic substance contained within a primary drug container and the secondary package delivery system, such as an autoinjector. In some embodiments, the secondary package may have a hydraulic medium that includes stabilizing chemical agents. The stabilizers can be the same as those incorporated into the drug (e.g., included in a drug formulation), e.g., for prolonging the shelf-life of the drug. The stabilizers can offer protection against damage caused by exposure to reactive agents, loss of volatile therapeutic, and reduction of excipients that cause degrative drug interactions. Exposure to reactive agents or loss of dissolved gasses over time can result in degradation and instability in the drug. Stabilizers can be included directly into the drug formulations to combat drug degradation via oxidation, but introduce their own degradation pathways that subsequently introduce independent degradation pathways and limit shelf life.

In some embodiments, systems and devices described herein can include stabilizing chemical agents, (e.g., sodium metabisulfite) around a container (e.g., a glass cartridge) containing a therapeutic substance (e.g., a drug), to capture reactive agents prior to their exposure to the therapeutic substance and enable reduction of stabilizing agents in the excipients of the therapeutic. The stabilizing agents can be provided in a liquid solution, a solid form, a coating, and/or another form. In some embodiments, systems and devices described herein can include a therapeutic substance delivery system that uses an intermediary fluid (e.g., a hydraulic or pressurized fluid) that interacts with a plunger of a drug container to deliver a therapeutic substance. In such embodiments, the intermediary fluid can include a stabilizer (or combination of stabilizers), e.g., to reduce degradation rate of the therapeutic substance. The intermediary fluid can include a stabilizer that is the same as the stabilizer included as an excipient in a therapeutic substance solution, e.g., to ensure compatibility of the stabilizers degradation byproducts with the specific therapeutic substance and control the migration of stabilizing agent byproducts via gas transmission or permeability rates through the primary drug container Alternatively, the intermediary fluid can include a stabilizing agent that is different from the stabilizing excipient included in the therapeutic substance solution. For example, the intermediary fluid may include a stabilizing agent that provides a higher degree of therapeutic protection but is not biocompatible and therefore not suitable for inclusion in the therapeutic substance solution as an excipient.

In some embodiments, therapeutic substance delivery system can be an auto-injector that provides a driving force, e.g., via a stored energy device and/or intermediary elements (e.g., a spring and an intermediary fluid), which applies pressure to a plunger of a therapeutic substance container or drug container. The container can be implemented as a glass cartridge and can include a glass cylinder, a plunger at a proximal end, and a seal (e.g., membrane) at a distal end. The container can store a therapeutic substance such as a drug. In some embodiments, the intermediary fluid containing the stabilizing agent can reduce the need for stabilizing excipients in the therapeutic substance or a solution containing the therapeutic substance and extending the shelf-life of the therapeutic substance, by reducing independent excipient degradation pathways. For example, the delivery system can include an intermediary fluid (e.g., a hydraulic or pressurized fluid) that can include sodium metabisulfite. Alternatively or additionally, the delivery system can include a housing or portions thereof that include an antioxidant or other stabilizing agent, such as, for example, a coating that includes an antioxidant or some other solid structure that includes an antioxidant. Still alternatively or additionally, one or more components of the therapeutic substance container of the delivery system can include an antioxidant or other stabilizing agent, e.g., the plunger can include an antioxidant, the seal can include an antioxidant, etc.

The stabilizing chemical agent can be any reducing agent that is incorporated into the secondary package e.g., an auto-injector to prevent degradation of the therapeutic in the primary drug container by capturing reactive elements prior to penetration into the primary drug container, modulating gas diffusion gradients through primary container components, and reduction of excipient levels. In some embodiments, the stabilizing agent can include sodium bisulfite or sodium metabisulfite. In some embodiments, the reducing agent can include ascorbic acid, alpha tocopherol, gamma tocopherol, and/or ammonia.

### DRAWINGS

FIG. 1 schematically depicts an example therapeutic delivery system, according to embodiments.

FIGS. 2A and 2B schematically depict different configurations of antioxidant in therapeutic delivery systems, according to embodiments.

FIGS. 3A and 3B schematically depict loss or depletion of antioxidants over time from a therapeutic substance.

FIG. 4 schematically depicts an example implementation of including antioxidants in an intermediary fluid of a therapeutic delivery system, according to embodiments.

FIG. 5 schematically depicts an example implementation of including antioxidants in a plunger of a therapeutic delivery system, according to embodiments.

FIG. 6 schematically depicts an example implementation of including antioxidants in or about a housing of a therapeutic delivery system, according to embodiments.

FIG. 7 depicts a perspective view of an example therapeutic delivery system, according to embodiments.

FIG. 8 depicts a side view of the therapeutic delivery system of FIG. 7, with a portion of the outer housing removed to show internal elements.

FIG. 9 depicts an exploded view of the therapeutic delivery system of FIG. 7.

FIG. 10 shows the degradation rates of sodium bisulfite in the primary drug container with a concentration driven expiration line at 0.7 mg/mL point below which the therapeutic is no longer stabilized. The circle trendline represents a standard formulation without NaSO3 in the hydraulic solution. The other two trendlines represent systems using the stabilizing solution at two different starting concentrations.

### DETAILED DESCRIPTION

Systems, devices, and methods are described herein for delivering a therapeutic substance to a patient, e.g., via a needle-based delivery system such as an auto-injection system. In some embodiments, systems, devices, and methods relate to therapeutic delivery systems are integrated with stabilizing agents or antioxidants for reducing oxidation degradation, loss of dissolved gas, and reduction of excipients in the primary drug container for the therapeutic substance.

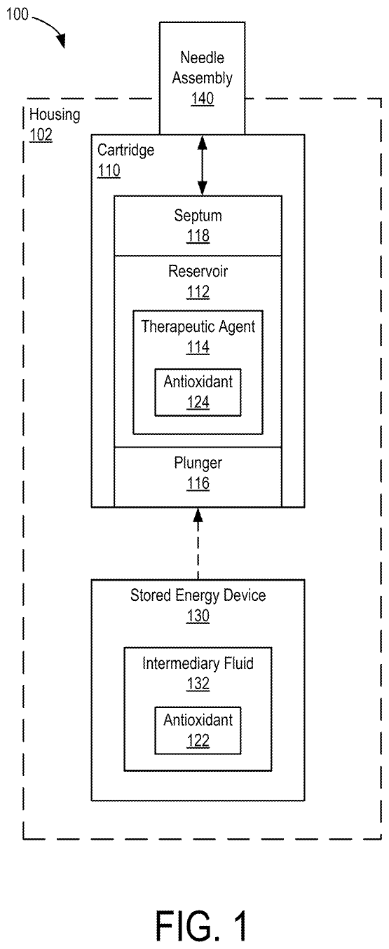

As schematically illustrated in FIG. 1, a therapeutic delivery system 100 can include a needle assembly 140, a cartridge or primary container 110, and a stored energy device 130, according to embodiments. Optionally, the therapeutic delivery system 100 can include a housing 102 that houses or contains one or more of the needle assembly 140, the cartridge 110, and the stored energy device 130. The therapeutic delivery system 100 can be any type of needle-based system or device for delivering a dose (e.g., an amount or volume) of a therapeutic substance. In some embodiments, the therapeutic delivery system 100 can be an auto-injector or auto-injection system, e.g., such as a device that is self-administered by a patient who is experiencing an emergency health condition. In some embodiments, the therapeutic delivery system 100 can be a miniaturized wearable injection device. In some embodiments, the therapeutic delivery system 100 can be configured to be a handheld device or be integrated into a handheld device.

The housing 102 can include one or more sections. In some embodiments, the housing 102 can include a main body and a removable cap that is configured to cover at least a portion of the main body. The removable cap can be configured to cover the portion of the main body so that a needle contained within the main body is shielded, e.g., to maintain sterility of the needle prior to use and/or to avoid accidental needle ejection. In some embodiments, the removable cap can also be placed over the main body such that an activation device or actuator for activating the therapeutic delivery system 100 is covered, e.g., to avoid accidental activation. In use, the removable cap of the housing 102 can be removed to enable the needle to be ejected into a patient's body. In some embodiments, the portion of the main body that is exposed once the removable cap is removed can be pressed or placed against a tissue surface, and an activation device (e.g., a button, switch, slider, knob, etc.) can be activated to cause the needle to eject out of the main body and through the tissue surface.

The needle assembly 140 can include a needle or cannula and a needle hub. The needle can include a sharp distal end that extends from the hub and is configured to penetrate through tissue. The needle hub can support the needle, e.g., relative to the cartridge 110, housing 102, and/or other portions of the therapeutic delivery system 100. In some embodiments, the needle may be held within the housing 102 (e.g., held within a main body of the housing 102) until an activation element is activated (e.g., a button, switch, or other activation device is pressed or actuated). In some embodiments, the needle can be a double-ended needle and include a proximal end that is also sharp. In use, the proximal end of the needle can be configured to puncture through a septum, membrane, or other seal 118.

The cartridge 110 can include a septum 118, a reservoir 112 that contains a therapeutic agent or substance 114, and a plunger 116. In some embodiments, the cartridge 110 can include a body that is formed of a rigid material, such as, for example, glass, plastic, metal, or some combination of such materials. For example, the cartridge 110 can include a glass container, e.g., formed of borosilicate glass. In some embodiments, the body can include a bottom heel and a barrel that extends to a shoulder and a neck. The body can have an inner surface and an outer surface, with a wall thickness extending between the inner and outer surfaces. The reservoir 112 can be bounded on one end by the septum 118 and on the other end by the plunger 116. When not in use, the septum 118, plunger 116, and body of the cartridge 110 contain the therapeutic agent 114 within the reservoir 112.

The therapeutic agent 114 can include at least one of a drug, a vaccine, a protein, a peptide, a gene, a compound or another pharmaceutically active ingredient. Examples of therapeutic agents suitable for use in the system 100 include glucagon, insulin, adrenaline, epinephrine, anti-venom, atropine, antibody formulations, antidotes to chemical agents, and the like. In some embodiments, the medication suitable for use in the device of the present invention is at least one medication selected from the group of medications identified by tradenames consisting of Acthar, Actimmune, Apokyn, AquaMephyton, Aranesp, Arixtra, Avonex, Betaseron, Bravelle, Butorphanol, Byetta, Calcijex, Calcitonin, Caverject, Cetrotide, Chorionic Gonadotropin, Cimzia, Copaxone, Copegus, DDAVP, D.H.E-45, Delatestryl, Delestrogen, Depo-Estradiol, Depo-Provera 150, Depo-SubQ Provera 104, Depo-Testosterone, Desmopressin, Dihydroergotamine, Edex, Eligard, Enbrel, Epipen, Epogen, Exjade, Faslodex, Fertinex, Follistim, Forteo, Fragmin, Fuzeon, Ganirelix acetate, Genotropin, Gleevec, Glucagon, Gonal, Heparin, Humatrope, Humira, Imitrex, Increlex, Infergen, Innohep, Insulin, Intron A, iPlex, Ketorolac, Kestrone, Kineret, Kuvan, Leukine, Leuprolide Acetate, Lovenox, Lupron, Luveris, Medroxyprogesterone, Menopur, Methotrexate, Miacalcin, Muse, Neumega, Neulasta, Neupogen, Nexavar, Norditropin, Novarel, Nutropin, NuvaRing, Omnitrope, Orfadin, Ovidrel, Pegasys, Peg-Intron, Pregnyl, Procrit, Profasi, Progesterone, Pulmozyme, Raptiva, Rebetol, Rebif, Repronex, Revlimid, Ribasphere, Ribavirin, Saizen, Sandostatin, Sensipar, Serostim, Somatuline, Sprycel, Somavert, Stadol, Sumatriptan, Supprelin, Sutent, Symlin, Tarceva, Testosterone, Temodar, Tev-Tropin, Thalomid, Tobi, Tykerb, Vitamin B12, Vitamin D, Vitamin K, Xeloda, Zemplar, and Zorbtive. In some embodiments, the system 100 is capable of delivering highly viscous therapeutic agents (e.g., having a viscosity greater than that of water, having a viscosity greater than 30 centipoise, having a viscosity between 30 centipoise and 50 centipoise, etc.), such as biologicals or monoclonal antibodies. In some embodiments, the therapeutic agent 114 can be incorporated in an aqueous solution. It will be apparent to those of skill in the art that the therapeutic agents listed above are only exemplary and that the system 100 is equally suitable for use in connection with practically any other injectable therapeutic agent not specifically listed herein.

The septum or seal 118 can function as a partition, enclosing or sealing off the therapeutic agent 114 from an external environment. In some embodiments, septum 118 can be implemented as a crimp cap seal or septum (e.g., a crimp camp combi-seal) that is to the body of the cartridge 110. The septum 118 can be formed of a flexible material, e.g., silicone, rubber, or other type of plastic. The septum 118 can be disposed at a distal or dispensing end of the cartridge 110. The dispensing end of the cartridge 110 can be configured to receive a needle hub of the needle assembly 140 thereon, such as, for example, the needle hub of a double-ended needle. The needle hub can be coupled to the dispensing end by any known technique, such as threaded connection or snap fit configurations. Once advanced onto the cartridge 110, the double-ended needle can pierce the septum 118 to dispense the therapeutic agent 114 from the cartridge 110.

In use, one end of a needle of the needle assembly 140 can be placed within the reservoir 112, e.g., after puncturing through the septum 118, and another end of the needle can be inserted into a patient's body. Fluid communication of the therapeutic agent 114 can then happen through the needle into the patient's body. The plunger 116 can slide along a length of the body of the cartridge 110, e.g., by sliding along the inner surface of the body of the cartridge 110. As the plunger 116 moves distally toward the needle, the plunger 116 can extrude an amount or volume of the therapeutic agent 114 through the needle and into the patient's body. The amount or volume extruded can be a predetermined or preset amount, e.g., dependent on the therapy or treatment being delivered to the patient.

The plunger 116 can be driven by energy imparted by a stored energy device 130. In some embodiments, the plunger 116 can be driven hydraulically, e.g., by an intermediary fluid 132 (e.g., a hydraulic or pressurized liquid and/or gas). Alternatively or additionally, the plunger 116 can be driven manually, e.g., via a user pressing down on a shaft, slider, or other mechanical actuator that is coupled to the plunger 116. Still alternatively or additionally, the plunger 116 can be driven mechanically, e.g., using a spring.

The stored energy device 130 can be configured to supply energy, e.g., to drive movement of the needle, the plunger 116, or other components of the therapeutic delivery system 110. In some embodiments, the stored energy device 130 can be configured to supply energy in response to being released or activated. For example, an activation device such as a button, tab, slider, knob, or other mechanism can be coupled to the stored energy device 130, and upon activation of such device, the stored energy device 130 can be configured to deliver stored energy to drive the delivery of the therapeutic agent 114. In some embodiments, the stored energy device 130 can include a deployment spring that drives a piston, which then delivers intermediary fluid 132 to the plunger 116. The deployment spring can be locked in a compressed configuration, and can be released upon actuation of an activation device. Alternatively, in some embodiments, the stored energy device 130 can include an electric motor to drive the piston. Still alternatively, in some embodiments, the stored energy device 130 can include pressurized fluid containers that can open to release an intermediary fluid 130. It can be appreciated that other suitable forms of energy storage and release can be used with the stored energy device 130 without departing from the scope of the present disclosure.

In some embodiments, the energy provided by a single stored energy device 130 can be used to drive needle ejection or insertion (e.g., via movement of the needle assembly 140) and to drive drug extrusion (e.g., via movement of the plunger 116). Alternatively, multiple stored energy devices 130 can be used, with each stored energy device 130 being used to drive a different component of the therapeutic delivery system 100. For example, a first stored energy device 130 can be configured to supply energy for driving needle ejection, while a second stored energy device 130 can be configured to supply energy for driving drug extrusion or plunger movement.

In some embodiments, the therapeutic delivery system 100 can include a throttling element, e.g., similar to that described with reference to International PCT Application No. PCT/US2023/061093, filed Jan. 23, 2023, titled “FLUIDIC THROTTLE CONTROL FOR THE DELIVERY OF STANDARD AND RHEOLOGICALLY CHALLENGING THERAPIES IN NEEDLE-BASED DRUG DELIVERY SYSTEMS,” the disclosure of which is incorporated herein by reference.

In some embodiments, the therapeutic agent 114 solution can include a stabilizing agent such as an antioxidant 124. For example, a liquid solution can include the therapeutic agent 114 (e.g., a drug, a vaccine, a protein, a peptide, a gene, a compound or another pharmaceutically active ingredient) and an antioxidant 124. The antioxidant 124 or other stabilizing agent can be configured to reduce oxidation and degradation of the therapeutic agent 114. For example, the antioxidant can be configured to slow the escape of hydrogen sulfide gas transmission through the primary container 112. In some embodiments, the antioxidant can include sodium bisulfite or sodium metabisulfite, but other suitable antioxidants can be used (e.g., ascorbic acid, alpha tocopherol, gamma tocopherol, ammonia, etc.).

Excipients such as sodium bisulfite or sodium metabisulfite have been used as antioxidants to prevent epinephrine degradation from chemical oxidation, e.g., in the approved epinephrine auto-injector products for anaphylaxis treatment. Due to the chemical ionization equilibria of these excipients, a gaseous hydrogen bisulfite (H₂S) can form, creating a pressure differential to atmosphere, and significantly migrate out from a low pH or acidic aqueous solution to the atmosphere, e.g., via the septum/glass cartridge joint and/or at the plunger/glass cartridge joint and/or through the semipermeable plunger material within the cartridge (e.g., septum 118 or plunger 116 of cartridge 110). Therefore, higher concentrations of these excipients may be necessary to compensate for their losses over time and to ensure their presence in the formulations, e.g., to protect epinephrine from oxidation and degradation throughout its shelf-life. Higher concentrations of these excipients, however, can also lead to degradation complications. For example, these excipients can also cross-react to epinephrine and cause a faster degradation rate resulting in shorter shelf-lives of the epinephrine product. In other words, higher concentrations of these excipients may slow the oxygen driven degradation of epinephrine but introduces its own degradation pathway. As such, while certain concentrations of excipients such as sodium bisulfite or sodium metabisulfite can be included in a therapeutic agent solution, these concentrations cannot exceed certain amounts.

As the antioxidants within the therapeutic agent are taken up by oxygen intrusion/migration, in some embodiments, to slow the loss or depletion of antioxidants such as, for example, sodium bisulfite or sodium metabisulfite, over time, the intermediary fluid 132 can include a reducing agent 122 that can act as an antioxidant and provide a favorable gas permeability diffusion pressure gradient. For example, the intermediary fluid 132 can include a concentration of antioxidant 122 that can reduce a degradation rate of the therapeutic agent 114 contained within the reservoir 112. In some embodiments, the cartridge 110 can be configured to be at least partially surrounded by or submersed in the intermediary fluid 132, which can contain the antioxidant 122. For example, a proximal end of the cartridge 110 including the plunger 116 can be surrounded by or submersed in the intermediary fluid 132. Alternatively or additionally, other portions of the cartridge 110 (e.g., a distal end of the cartridge 110 including the septum 118, and/or intermediate portions of the cartridge 110) can be surrounded by or submersed in the intermediary fluid 132. The intermediary fluid 132 can include a high concentration of the antioxidant 122. This antioxidant 122 can function to react with oxygen intruding into the intermediary fluid, thus making less oxygen available to pass the plunger/glass cartridge joint and/or the septum/glass cartridge joint depending on embodiment. As there is less oxygen intrusion into the cartridge, the antioxidant is preserved therefore extending the shelf life of the therapeutic agent. In particular, because the intermediary fluid 132 does not contain a therapeutic agent nor is it formulated for delivery into a patient, the intermediary fluid 132 can incorporate a greater concentration of the antioxidant 122.

In some embodiments, with the intermediary fluid 132 including an antioxidant, the concentration of the antioxidant or other excipients in the therapeutic agent 114 solution can be reduced, e.g., to reduce the degradation of the therapeutic agent 114 due to cross-reactions between the therapeutic agent 114 and the excipients. As such, in some embodiments, the concentration of the antioxidant in the therapeutic agent 114 can be reduced by about 10% to about 50%, including all sub-ranges and values therebetween, when an antioxidant is included in the intermediary fluid 132. In some embodiments, the reduction of the concentration of the antioxidant in the therapeutic agent 114 solution can be related to the concentration of the antioxidant in the intermediary fluid 134, e.g., such that the concentration of antioxidant in the intermediary fluid 132 is a certain factor greater than the antioxidant in the therapeutic agent 114 solution, or that a total amount of antioxidant in the intermediary fluid 132 and in the therapeutic agent 114 solution is achieved. The concentrations of antioxidants in the therapeutic and intermediary fluid can be manipulated in such a way that byproducts of oxygen and antioxidant reaction, for example H₂S, can create a pressure differential between the intermediary fluid and the therapeutic fluid where the pressure is greater in the intermediary fluid. As this pressure differential will drive said byproducts past the plunger/glass cartridge joint, the pressure in the therapeutic fluid will be increased to some pressure above atmospheric pressure. This pressure differential between atmospheric and therapeutic fluid will prevent oxygen intrusion into the therapeutic fluid past the septum/glass cartridge joint.

In some embodiments, the intermediary fluid 132 can include between about two times and about 20 times the concentration of antioxidant as the therapeutic agent 114 solution, including all sub-ranges and values therebetween. In some embodiments, the intermediary fluid 132 can include at least about 3 times, at least about 4 times, at least about 5 times, or at least about 6 times the concentration of the antioxidant in the therapeutic agent 114 solution. For example, the intermediary fluid 132 can include between about 4 and about 5 times the concentration of antioxidant as the therapeutic agent 114 solution, including all sub-ranges and values therebetween.

In some embodiments, the antioxidant 124 in the therapeutic agent 114 solution and the antioxidant 122 in the intermediary fluid 132 can be the same antioxidant. For example, the antioxidant 122 can be selected to be the same as the antioxidant 124 such that the interactions between the antioxidant 122 and the therapeutic agent 114 are known, e.g., for ensuring compatibility between the antioxidant 122 and the therapeutic agent 114 or for avoiding complications (e.g., unexpected reactions) between the antioxidant 122 and the therapeutic agent 114. Alternatively, the antioxidant 124 in the therapeutic agent 114 solution can be different from the antioxidant in the intermediary fluid 132. For example, in some embodiments, a stabilizer that provides greater protection against multiple routes of degradation or effectiveness can be selected as the agent 122, which can differ from the antioxidant 124 in the therapeutic agent 114 solution, e.g., due to toxicity and/or drug-excipient compatibility reasons and/or opacity.

By having an intermediary fluid 132 with one or more stabilizers 122, the shelf-life of the therapeutic agent 114 can be increased by about 10% to about 500%, including all sub-ranges or values therebetween, including, for example, about 100% (or about doubling the shelf-life). For example, the intermediary fluid 132 with the stabilizers can be configured to increase the shelf-life of the therapeutic agent by at least about a factor of 2. In some embodiments, with a concentration of antioxidant in the intermediary fluid 132 being about 4 to about 5 times that of the standard amount of antioxidant in the therapeutic agent 114 solution, the antioxidant in the therapeutic agent can be decreased by 75% and the resultant shelf-life of the therapeutic agent 114 can be increased by at least about a factor of 2, e.g., 18 months increased to 3 years.

In some embodiments, certain components of needle-based injection systems as described herein can be similar to those described in U.S. Patent Application Publication No. 2021/0386932, published Dec. 16, 2021, titled “Miniaturized wearable medication administration device,” and PCT Patent Application Publication No. WO2022061221, published Mar. 24, 2022, titled “Protective sheath for a glass pharmaceutical cartridge, system, and method of manufacture,” the disclosures of each of which are incorporated herein by reference.

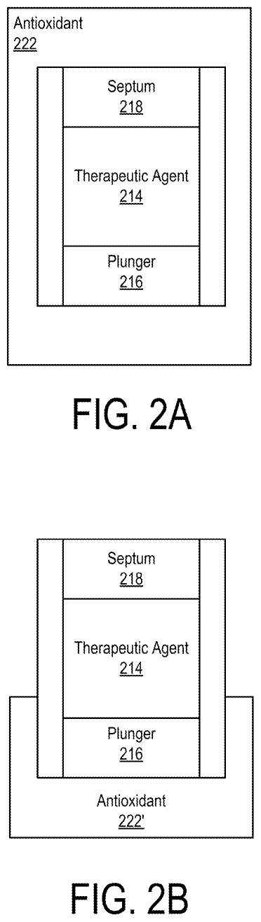

FIGS. 2A and 2B depict different configurations of intermediary fluid with stabilizing agents being disposed about or around a cartridge of therapeutic delivery system, according to embodiments. In FIGS. 2A and 2B, the cartridge can include a plunger 216 and a septum 218 and can contain a therapeutic agent 214. The plunger 216, the septum 218, and the therapeutic agent 214 can be structurally and/or functionally similar to the plunger 116, the septum 118, and the therapeutic agent 114 described with reference to FIG. 1. According, certain details of the plunger 216, the septum 218, and the therapeutic agent 214 are not repeated herein.

The cartridge can include a plunger 216 at a proximal end and a septum 218 at a distal end. Between the plunger 216 and the septum 218 can be a reservoir that contains the therapeutic agent 214. In some embodiments, the therapeutic agent 214 can be provided in an aqueous solution or other type of liquid solution. In some embodiments, the therapeutic agent 214 can be sensitive to oxidation. Oxidation can be caused by, for example, oxygen or other oxidizing substances coming into contact with the therapeutic agent 214. Oxygen or other oxidizing substances can pass into the reservoir and interact with the therapeutic agent 214 via the plunger 216 and/or the septum 218. As such, in some embodiments, the therapeutic agent 214 (or a solution containing the therapeutic agent 214) can include an antioxidant, e.g., similar to that described with reference to therapeutic agent 114. The antioxidant can be configured to reduce oxidation of the therapeutic agent 214, e.g., to reduce degradation of the therapeutic agent 214.

Additionally or alternatively, an antioxidant can be included in the drug delivery system outside of the therapeutic agent 214 solution. For example, the cartridge can be surrounded by or submerged in a protective fluid for example, an opaque solution containing an antioxidant that generates a H₂S vapor pressure, to provide light protection, prevent oxygen penetration and prevent therapeutic H₂S loss. In some embodiments, an stabilizing fluid 222 can be configured to surround both ends or the entirety of the cartridge, including the proximal end including the plunger 216 and the distal end including the septum 218, as shown in FIG. 2A. Alternatively, in some embodiments, a stabilizing fluid 222′ can be configured to surround a portion of the cartridge, e.g., the proximal end including the plunger 214, as shown in FIG. 2B.

In some embodiments, the antioxidant 222, 222′ can be incorporated into an intermediary fluid, such as, for example, a hydraulic fluid of a hydraulic fluid driven delivery system, as described above with reference to FIG. 1. In some embodiments, the stabilizing agent 222, 222′ can be included in a coating, e.g., a coating that covers at least a portion of the plunger 216 and/or the septum 218, or a coating that covers an inner surface (or portion thereof) of the housing defined by the drug delivery system. In some embodiments, the stabilizing agent 222 can be included in a solid structure such as an outer housing, an inner housing defining one or more chambers, etc. In some embodiments, the antioxidant 222, 222′ can be a gas, solid, or liquid that is included or contained within one or more chambers or spaces defined within a drug delivery system.

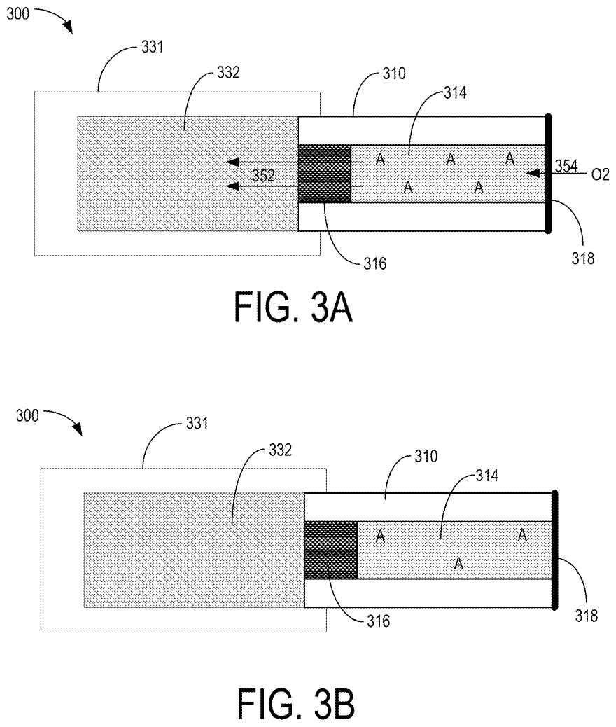

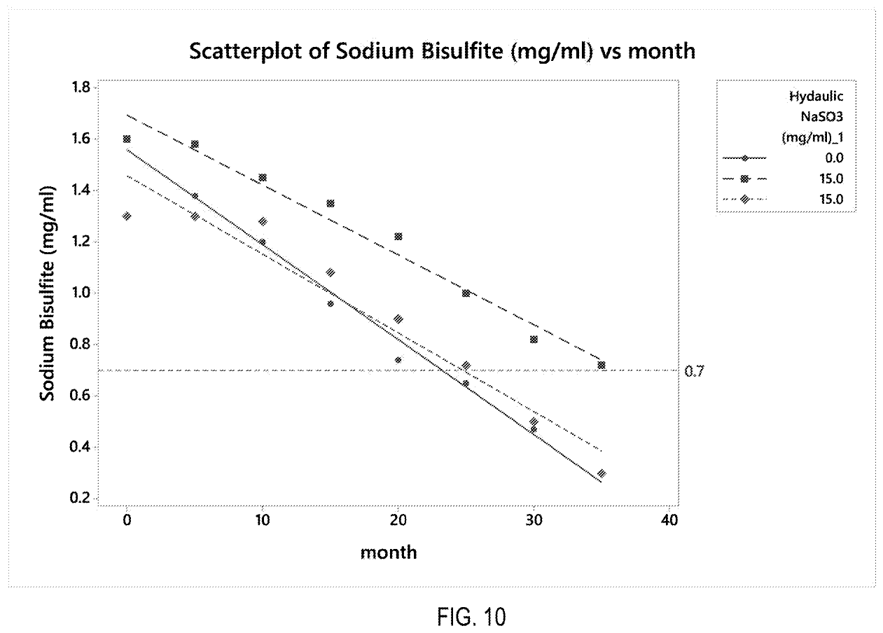

FIGS. 3A, 3B and 10 depict the loss or depletion of an antioxidant within a therapeutic substance 314 solution over time in a therapeutic delivery system 300, according to embodiments. As shown in FIG. 3A, a therapeutic substance 314 solution can include a starting concentration of an antioxidant A, as schematically represented by the A's in the therapeutic substance 314 solution. Over time, the derivatives, such as hydrogen sulfide, of antioxidant A can migrate out of the therapeutic substance 314 solution.

In particular, the therapeutic delivery system 300 can be used for delivery of the therapeutic substance 314, e.g., via a needle injection. The therapeutic delivery system 300 can be structurally and/or functionally similar to other therapeutic delivery systems described herein, including, for example, therapeutic delivery system 100. For example, the therapeutic delivery system 300 can include a cartridge 310 having a plunger 316 and a septum 318, and a housing 331 that contains an intermediary fluid 332. While not depicted, the therapeutic delivery system 300 can also include a stored energy device (e.g., stored energy device 130), a needle assembly (e.g., needle assembly 140), a throttling element, and/or other elements described above with respect to therapeutic delivery system 100.

As schematically depicted in FIG. 3A, the derivatives of antioxidant A in the therapeutic substance 314 solution can migrate, via gas rubber permeability, out of the therapeutic substance 314 solution and/or become depleted, resulting in more rapid consumption of antioxidant A. In one instance, the derivatives of antioxidant A of the therapeutic substance 314 solution via permeation through the plunger 316. The plunger 316 can be disposed adjacent to the intermediary fluid 332. If the intermediary fluid 332 does not include any pressure gradient, then a diffusion gradient would exist between the intermediary fluid 332 and the therapeutic substance 314 solution. Over time, the higher concentration of antioxidant A in the therapeutic substance 314 solution may migrate out of the therapeutic substance 314 solution, through the plunger 316, and into the intermediary solution 332, as schematically represented by arrows 352. In another instance, oxygen O that permeates through the plunger 316 into the cartridge, as schematically illustrated via arrow 354, and interacts with the antioxidant A in the therapeutic substance 314 solution can deplete the antioxidant A over time.

In some embodiments, due to the chemical ionization equilibrium of the antioxidant A, a gaseous form of the antioxidant A can form and migrate out of the therapeutic substance 314 solution. For example, sodium bisulfite or sodium metabisulfite are commonly used antioxidants in epinephrine delivery systems, e.g., to reduce or present oxidation of epinephrine. Due to the chemical ionization equilibria of sodium bisulfite or sodium metabisulfite, a gaseous hydrogen bisulfite (H₂S) can form, creating a pressure differential between the therapeutic fluid and atmosphere, enabling significantly migration out from the low pH aqueous solution to the atmosphere, via the septum 318. Such migration increased the rate of consumption of the antioxidant A in the therapeutic substance 314 solution over time.

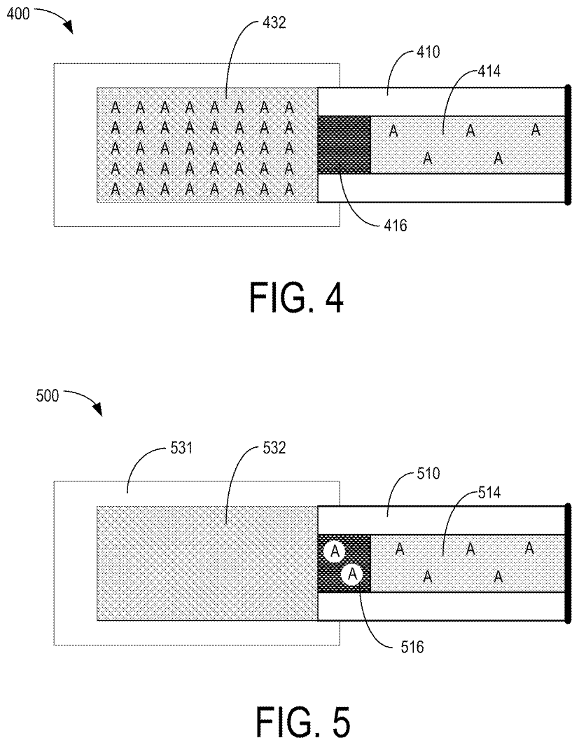

Systems, devices, and methods described herein can provide various solutions for combating the migration and/or depletion of antioxidant A over time. FIG. 4 depicts a solution where an antioxidant A is incorporated into an intermediary fluid 432 of a therapeutic delivery system 400, according to embodiments. The therapeutic delivery system 400 can be structurally and/or functionally similar to other therapeutic delivery systems described herein, including, for example, therapeutic delivery system 100, 300, etc. For example, the therapeutic delivery system 400 can include a cartridge 410 that includes a plunger 416 and a therapeutic substance 414. The therapeutic substance 414 can be contained within a solution that includes a first concentration of an antioxidant A. The cartridge 410 can be disposed adjacent to an intermediary fluid 432, e.g., for driving the delivery of the therapeutic substance 414, as described with reference to FIG. 1. The intermediary fluid 432 can include a second concentration of an antioxidant A, where the second concentration is greater than the first concentration. By having the intermediary fluid 432 include a concentration of antioxidant A that is greater than that of the therapeutic substance 414 solution, the system 400 can avoid migration of the antioxidant A out of the therapeutic substance 414 solution due to a chemical gradient from the therapeutic substance 414 solution to the intermediary solution 432. Additionally, this will create a favorable direction of permeability from the intermediary fluid through the plunger and lastly through the crimp cap. This advantageously creates a slower oxygen permeation through the crimp cap into the therapeutic solution.

FIG. 5 depicts another solution for reducing depletion or migration of antioxidant A, where an antioxidant A is incorporated into a plunger 516 of a cartridge 510 of a therapeutic delivery system 500, according to embodiments. FIG. 10 shows the depletion rate graphically. The therapeutic delivery system 500 can be structurally and/or functionally similar to other therapeutic delivery systems described herein, including, for example, therapeutic delivery system 100, 300, 400, etc. For example, the therapeutic delivery system 500 includes the cartridge 510 having the plunger 516 and the therapeutic substance 514. An antioxidant A can be incorporated into the plunger 516. For example, the plunger 516 can be formed of a material that has antioxidative properties. Alternatively or additionally, the plunger 516 can be coated (entirely or partially) by an antioxidant. By having the plunger 516 include an antioxidant A, the migration and/or depletion of antioxidant A from the therapeutic substance 514 solution can be reduced. In particular, the antioxidant A in the therapeutic substance 514 solution may not migrate out of the therapeutic substance 514 solution via the plunger 516 because the plunger 516 includes an antioxidant A that negates or reduces the effect of any chemical gradient between the therapeutic substance 514 solution and an intermediary solution 532 on the other side of the plunger 516. Oxygen or other oxidative substances that might otherwise pass through the plunger 516 and into the therapeutic substance 514 solution would also be captured by the antioxidant A in the plunger 516 before coming into contact with the therapeutic substance 514 solution.

While not depicted in FIG. 5, it can be appreciated that the antioxidant A can also be incorporated into other elements of the delivery system 500. For example, similar to the delivery system 400, the intermediary solution 532 can include an antioxidant A. Antioxidant A can also be incorporated into the septum of the cartridge 510, the cylinder of the cartridge 510, and/or other elements of the system 500.

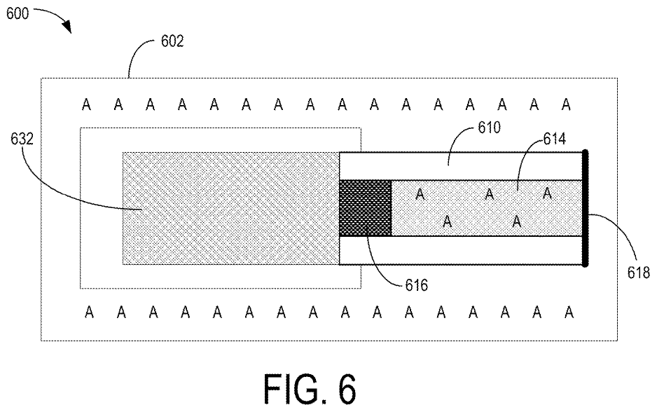

FIG. 6 depicts another solution for reducing depletion or migration of antioxidant A, where an antioxidant A is incorporated into a housing 602 of a therapeutic delivery system 600, according to embodiments. The therapeutic delivery system 600 can be structurally and/or functionally similar to other therapeutic delivery systems described herein, including, for example, therapeutic delivery system 100, 300, 400, 500, etc. For example, the therapeutic delivery system 600 includes the cartridge 610 having a plunger 616, a septum 618, and a therapeutic substance 614. The cartridge 610 can be disposed adjacent to an intermediary fluid 632, e.g., for driving the delivery of the therapeutic substance 614, as described with reference to FIG. 1. The therapeutic substance 414 can be contained within a solution that includes a concentration of an antioxidant A. The housing 602 of the delivery system 600 can also include an antioxidant A, e.g., incorporated into the housing 602 material, coated on the housing 602, and/or disposed around or about the housing 602 (e.g., in one or more compartments or spaces defined by the housing 602). The antioxidant A included n the housing 602 can reduce the depletion of the antioxidant A in the therapeutic substance 614 solution, e.g., by capturing and reducing oxygen within the system 600. In some embodiments, the housing 602 can form a fluid tight seal around other internal components of the delivery system 600. In such embodiments, the housing 602 can contain a liquid, gas, or solid antioxidant A that can capture and reduce oxygen within the system 600, e.g., before that oxygen contacts with the therapeutic substance 614 solution.

While not depicted in FIG. 6, it can be appreciated that the antioxidant A can also be incorporated into other elements of the delivery system 600. For example, similar to the delivery system 400, the intermediary solution 632 can include an antioxidant A. Antioxidant A can also be incorporated into the septum or plunger of the cartridge 610 and/or other elements of the system 600.

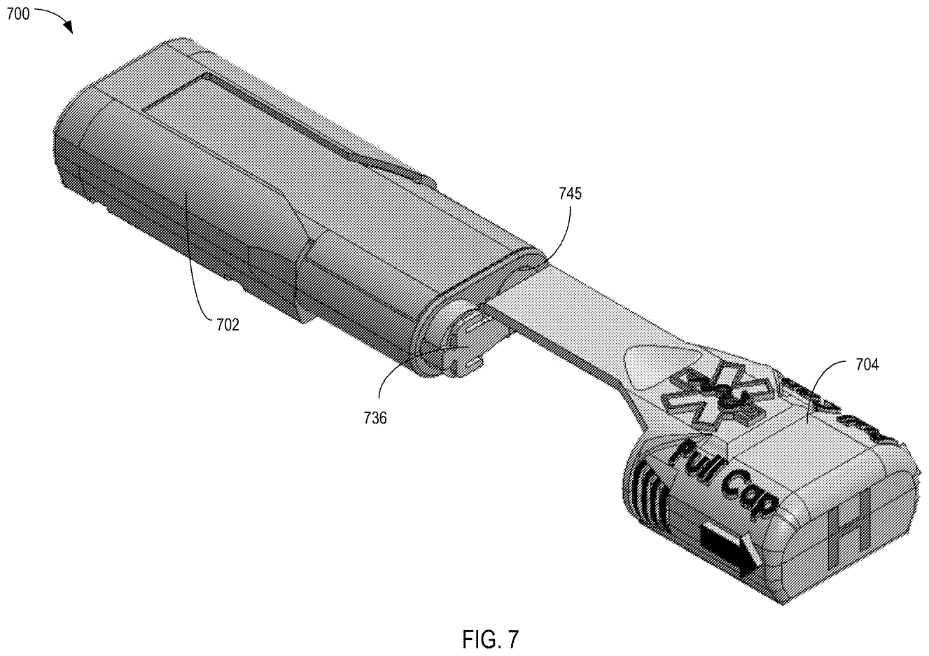

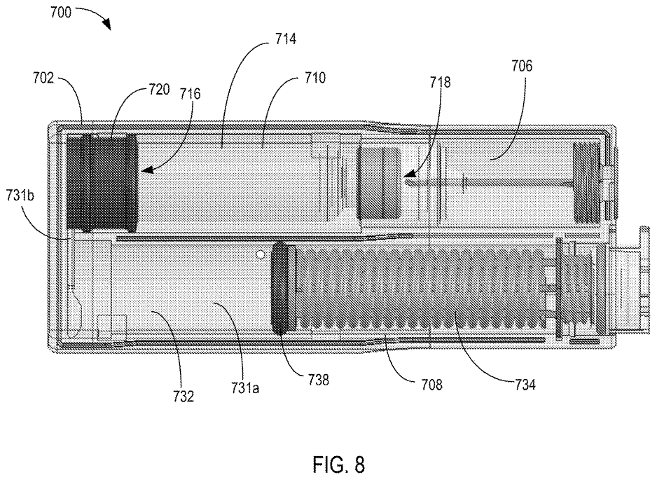

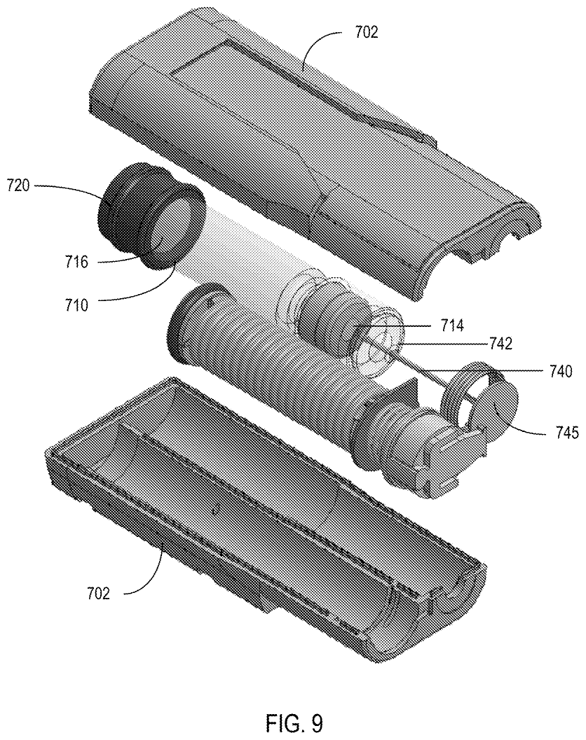

Referring now to FIG. 7-9, an example of a therapeutic delivery system 700 is described. FIG. 7 depicts a schematic view of the therapeutic delivery system 700, FIG. 8 depicts a side view of the therapeutic delivery system 700 with a portion of an outer housing 702 removed to show internal components, and FIG. 9 depicts an exploded view of the therapeutic delivery system 700. The therapeutic delivery system 700 can be structurally and/or functionally similar to other therapeutic delivery systems described herein, including, for example, therapeutic delivery systems 100, 300, 400, 500, 600, etc. As shown in FIGS. 7-9, the therapeutic delivery system 700 can be a needle-based injection system.

The system 700 can include a main body or housing 702 and a cap 704. The housing 702 can contain other components of the system 700, including, for example, a cartridge 710 including a plunger 716 and a septum 718, a stored energy device including a spring 734, a needle assembly 740 including a hub 742, a throttling element, etc. These elements can be similar to those of other delivery systems described herein, e.g., including, delivery system 100, and therefore certain details of these elements are not repeated in detail herein again. The cap 704 can be configured to cover a portion of the therapeutic delivery system 700. In FIG. 7, the cap 704 is shown removed from the housing 702, e.g., to expose one or more other components of the therapeutic delivery system 700. In particular, the removal of the cap 704 can expose an activation device (e.g., a button) and a septum 745 that covers an opening through which a needle of the needle assembly 740 can be ejected out of the housing 702.

Prior to use, the spring 734 of the stored energy device can be in a compressed or undeployed state. The spring 734 can occupy a space or compartment 708 within the housing 702. The spring 734 can be coupled to a plunger 738, which can be adjacent to an intermediary fluid 732. The intermediary fluid 732 can be disposed within a space 731 (including cylindrical channel 731a and a passageway 731b extending to a region adjacent to the plunger 716 of the cartridge 710) within the housing 702. A portion of the intermediary fluid 732 can be disposed adjacent to a throttling element and/or the plunger 716. The cartridge 710 can include the plunger 716 at is proximal end and the septum 718 at its distal end. The cartridge 710 can contain a therapeutic substance 714, e.g., in a cylindrical space between the plunger 716 and the septum 718. The cartridge 710 can be disposed upstream or proximal of the needle assembly 740. The needle assembly 740 can occupy a space 706 within the housing 702.

In some embodiments, the therapeutic substance 714 can be sensitive to oxidation. As such, to prevent or reduce degradation of the therapeutic substance 714 (and to increase shelf-life of the therapeutic substance 714), one or more antioxidants can be included in the system 700. In some embodiments, the intermediary fluid 732 can include an antioxidant. In some embodiments, the therapeutic substance 714 solution can include an antioxidant. In some embodiments, the concentration of antioxidant in the therapeutic substance 714 solution can be less than the concentration of antioxidant in the intermediary solution, e.g., to prevent a positive chemical gradient between the therapeutic substance 714 solution and the intermediary solution 732. In some embodiments, an antioxidant can be included in one or more locations within the housing 702 of the system 700. For example, an antioxidant can be included in one or more of spaces 708, 731a,b, and 706. In such embodiments, the antioxidant can be in liquid, gas, or solid form. For example, the housing 702 can form a fluid tight seal around the interior elements of the system 700, and within that seal, a gaseous, liquid, or solid antioxidant can be disposed. In some embodiments, the antioxidant can be a coating, e.g., applied to one or more elements of the system 700 such as, for example, one or more interior surfaces of the housing 702, the spring 734, the plunger 738, the throttling element 720, the plunger 716, the septum 718, the needle hub 742, and/or the septum 745.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within +10% of the recited value. For example, in some instances, “about 100 [units]” may mean within +10% of 100 (e.g., from 90 to 110). The terms “about” and “approximately” may be used interchangeably.

“A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

1. An apparatus, comprising:
a needle having a proximal end and a distal end;
a primary container having a body, a plunger, and a seal, the body, the plunger, and the seal collectively defining a reservoir configured to contain a therapeutic agent solution;
an intermediary fluid including a stabilizing agent configured to slow an escape of hydrogen sulfide gas transmission through at least a portion of the primary container;
a stored energy device configured to apply pressure to the intermediary fluid; and
an activation device configured to activate the stored energy device to apply pressure to the intermediary fluid such that the intermediary fluid acts on the primary container to drive a movement of the plunger to extrude the therapeutic agent solution out through the needle.

2. The apparatus of claim 1, wherein the stabilizing agent includes at least one of: sodium bisulfite or sodium metabisulfite.

3. The apparatus of claim 1, wherein the stabilizing agent has a first gas transmission rate, and the therapeutic agent solution includes a therapeutic antioxidant with byproducts that have a second gas transmission rate.

4. The apparatus of claim 3, wherein the first gas transmission rate and the second gas transmission rate are the same.

5. The apparatus of claim 3, wherein the stabilizing agent and the therapeutic antioxidant are the same.

6. The apparatus of claim 3, wherein the intermediary fluid includes a first concentration of the stabilizing agent, and the therapeutic agent solution includes a second concentration of the therapeutic antioxidant, the second concentration of the therapeutic antioxidant being less than the first concentration of the stabilizing agent.

7. The apparatus of claim 3, wherein the intermediary fluid and the therapeutic agent solution include respective concentrations of the stabilizing agent and the therapeutic antioxidant that are configured to produce byproducts in the intermediary fluid and the therapeutic agent solution that create a pressure differential between the intermediary fluid and the therapeutic fluid.

8. The apparatus of claim 7, wherein the pressure differential is configured to drive at least a portion of the byproducts past the plunger and into the primary container such that a pressure of the therapeutic fluid solution increases relative to atmospheric pressure.

9. (canceled)

10. (canceled)

11. An apparatus, comprising:
a needle having a proximal end and a distal end;
a primary container having a body, a plunger, and a seal, the body, the plunger, and the seal collectively defining a reservoir configured to contain a therapeutic agent solution;
an intermediary fluid including a stabilizing chemical agent to slow a rate of oxygen penetration into the therapeutic agent solution via gas transmission through at least a portion of the primary container;
a stored energy device configured to apply pressure to the intermediary fluid; and
an activation device configured to activate the stored energy device to apply pressure to the intermediary fluid such that the intermediary fluid acts on a cartridge to drive a movement of the plunger to extrude the therapeutic agent solution out through the needle.

12. (canceled)

13. (canceled)

14. (canceled)

15. (canceled)

16. (canceled)

17. (canceled)

18. (canceled)

19. An apparatus, comprising:
a needle having a proximal end and a distal end;
a primary container having a body, a plunger, and a seal, the body, the plunger, and the seal collectively defining a reservoir configured to contain a therapeutic agent solution;
an intermediary fluid including a stabilizing chemical agent configured to increase a shelf-life of the therapeutic agent solution;
a stored energy device configured to apply pressure to the intermediary fluid; and
an activation device configured to activate the stored energy device to apply pressure to the intermediary fluid such that the intermediary fluid acts on a cartridge to drive a movement of the plunger to extrude the therapeutic agent solution out through the needle.

20. (canceled)

21. The apparatus of claim 19, wherein the stabilizing agent has a first gas transmission rate, and the therapeutic agent solution includes a therapeutic antioxidant with byproducts that have a second gas transmission rate.

22. The apparatus of claim 21, wherein the first gas transmission rate and the second gas transmission rate are the same.

23. The apparatus of claim 21, wherein the stabilizing agent and the therapeutic antioxidant are the same.

24. The apparatus of claim 21, wherein the intermediary fluid includes a first concentration of the stabilizing agent, and the therapeutic agent solution includes a second concentration of the therapeutic antioxidant, the second concentration of the therapeutic antioxidant being less than the first concentration of the stabilizing agent.

25. The apparatus of claim 21, wherein the intermediary fluid and the therapeutic agent solution include respective concentrations of the stabilizing agent and the therapeutic antioxidant that are configured to produce byproducts in the intermediary fluid and the therapeutic agent solution that create a pressure differential between the intermediary fluid and the therapeutic fluid.

26. The apparatus of claim 25, wherein the pressure differential is configured to drive at least a portion of the byproducts past the plunger and into the primary container such that a pressure of the therapeutic fluid solution increases relative to atmospheric pressure.

27. The apparatus of claim 24, wherein the first concentration of the stabilizing agent in the intermediary fluid is between about four to about five times the first concentration of the therapeutic antioxidant in the therapeutic agent solution, the intermediary fluid with the first concentration of the stabilizing agent being configured to increase the shelf-life of the therapeutic agent by at least about a factor of 2.

28. (canceled)
